(19) United States
(12) Patent Application Publication
(10) Pub. No.: US 2023/0301512 A1
(43) Pub. Date: Sep. 28, 2023

(54) PORTABLE RETINA MEASURING DEVICE, AND OPHTHALMOLOGIC DISEASE MEASURING SYSTEM AND OPHTHALMOLOGIC DISEASE MANAGEMENT METHOD USING PORTABLE TERMINAL

(71) Applicant: AIFORPET, Pohang-si (KR)

(72) Inventors: Eu Na HEO, Seoul (KR); Eunsim HEO, Seoul (KR); Woojin HEO, Seoul (KR)

(21) Appl. No.: 18/019,799

(22) PCT Filed: Oct. 21, 2020
(86) PCT No.: PCT/KR2020/014380

§ 371 Date: Feb. 4, 2023

(30) Foreign Application Priority Data
Aug. 7, 2020 (KR) 10-2020-0099373
Aug. 7, 2020 (KR) 10-2020-0099375

(51) Int. Cl. A61B 3/14; A61B 3/12

(52) U.S. Cl. CPC A61B 3/14; A61B 3/1208; A61B 3/0041

(57) ABSTRACT

Disclosed are a portable retina measuring device, and an ophthalmologic disease measuring system and an ophthalmologic disease management method using the portable terminal. The portable retina measuring device comprises: a photographing unit which obtains a photographed image by photographing the retina of a photography subject; and a device control unit which uses the obtained photographed image to generate measurement result data, wherein the photography unit comprises a flat light source irradiation unit, the flat light source irradiation unit comprising: a light source unit that generates a light source for the photography subject; a grid that is located so as to face the light source unit and has a grid pattern; and a photographing element that is located in the center of the grid.

### TECHNICAL FIELD

The present invention relates to a portable retina measuring device, and an ophthalmologic disease measuring system and management method using a portable terminal, that is, a portable retina measuring device for measuring an ophthalmologic disease using a high-resolution video image acquired by imaging a retina of a target of imaging, for example, a companion animal, and an ophthalmologic disease measuring system and management method using a portable terminal for quickly determining whether a target has an ophthalmologic disease at an early stage when the ophthalmologic disease occurs, and rapidly treating the target.

### BACKGROUND ART

With the aging population structure and the increasing number of single-person households, human beings are gradually becoming self-centered and desolate. Accordingly, the number of people who recognize pets as family members or companions is increasing, and the companion animal market is also steadily growing.

As interest in diseases of such companion animals is increasing day by day, production and development of devices and methods for solving these problems are also increasing day by day.

However, information or devices for ophthalmologic diseases, a type of fatal disease that interferes with life span not only for humans but also for companion animals has not been sufficiently developed, and research on this subject is still poor. Accordingly, when a companion animal suffers from an incurable disease, such as the above ophthalmologic diseases, treatment is difficult and expensive, and thus there is a problem that it is virtually impossible to treat the companion animal (see Korean Patent Publication Nos. 2015-0111724 and 2015-0115980).

When these companion animals have abnormal symptoms that are not usually seen, most of the companion animals are taken to a veterinary hospital for treatment, or the abnormal symptoms that occur in the companion animals are resolved on the basis of information obtained from nearby people or through the Internet, phone calls, etc.

However, information obtained from nearby people or through the Internet, phone calls, etc. is incorrect in many cases, and thus people may have difficulty in treatment. Also, when people personally visit hospitals, there are many cases in which the waiting time at the hospital is long and the service is not properly provided to the customers due to the heavy hospital workload (see Korean Patent Publication No. 2003-0032088 (Apr. 26, 2003)).

### DETAILED DESCRIPTION OF THE INVENTION

### Technical Problems

The present invention is directed to providing a portable retina measuring device.

The present invention is also directed to providing an ophthalmologic disease measuring system and management method using a portable terminal.

Objectives of the present invention are not limited to those described above, and other objectives which have not been described will be clearly understood by those of ordinary skill in the art from the following description.

### Technical Solution

One aspect of the present invention provides a portable retina measuring device including an imaging unit configured to acquire a photographic image by imaging a retina of a target of imaging and a device control unit configured to generate measurement result data using the acquired photographic image. The imaging unit includes a plate-shaped light-source emitting unit including a light source unit configured to generate a light source for the target of imaging, a grid located to face the light source unit and having a grid pattern, and an imaging element located at a center of the grid.

The device control unit may adjust an imaging time using a distance measurement value between the target of imaging and the imaging unit.

The device control unit may adjust a lens focus value of the imaging element in accordance with a position of the retina of the target of imaging on the basis of the grid pattern.

When the distance measurement value and the lens focus value are both satisfactory, the device control unit may acquire a plurality of photographic images and generate the measurement result data including a high-resolution video image by processing and correcting the acquired photographic images on the basis of the grid pattern.

The photographic images may include a reference image acquired centering on the retina of the target of imaging and sub-images acquired from the periphery of the retina of the target of imaging.

The portable retina measuring device may further include a device management server configured to generate standard result data on the basis of the measurement result data and generate interpretation result data regarding the retina of the target of imaging by comparing and analyzing the standard result data and the measurement result data.

The portable retina measuring device may further include a sound generator configured to generate, when the target of imaging is a companion animal, a sound related to the companion animal.

Another aspect of the present invention provides an ophthalmologic disease measuring method using a portable terminal and performed by a healthcare server, the ophthalmologic disease measuring method including matching basic ophthalmology state information to basic ophthalmology result data to generate ophthalmologic health standard data, receiving ophthalmologic state measurement information of a test subject from a user terminal, and comparing and analyzing the ophthalmologic state measurement information on the basis of the ophthalmologic health standard data to generate ophthalmologic health result data. The generation of the ophthalmologic health standard data includes preprocessing images included in basic ophthalmology imaging information included in the basic ophthalmology state information, extracting analysis images from the preprocessed images, extracting part-specific analysis images from the analysis images and diagnosing whether there is an ophthalmologic disease in the part-specific analysis images to generate basic diagnosis data, determining disease-specific progression stages for the part-specific analysis images on the basis of the basic diagnosis data to generate basic determination data, and generating the basic ophthalmology result data including the basic diagnosis data and the basic determination data corresponding to the basic diagnosis data.

The healthcare server may simultaneously analyze the part-specific analysis images on the basis of basic information of the test subject included in the basic ophthalmology state information to classify the basic ophthalmology imaging information according to the disease-specific progression stages.

The acquiring of the basic ophthalmology imaging information may include imaging an eye of the test subject in a general imaging mode to acquire general imaging information and imaging the eye of the test subject in a scanner imaging mode in which a scanner is used so that a pupil of the test subject is scanned in a grid pattern, to acquire scanner imaging information. Here, the basic ophthalmology imaging information may include a photograph or a video.

The ophthalmologic disease measuring method may further include a filtering operation of determining, when the basic ophthalmology imaging information is the video, normal images from the video and extracting at least ten images.

When it is determined that there is an abnormal symptom in a corneal surface using the grid pattern in which the pupil of the test subject is scanned, the healthcare server may diagnose the test subject with the ophthalmologic disease and determine a disease-specific progression stage for the test subject.

The generation of the ophthalmologic health standard data may include repeatedly learning the basic ophthalmology result data corresponding to the basic ophthalmology state information to verify the basic ophthalmology result data.

The generation of the ophthalmologic health result data may include extracting an actual analysis image from actual ophthalmology imaging information included in the ophthalmologic state measurement information of the test subject, extracting part-specific actual analysis images from the actual analysis image to diagnose whether there is an ophthalmologic disease in the part-specific actual analysis images and generate actual ophthalmology diagnosis data, determining a disease-specific progression stage for the part-specific actual analysis images on the basis of the actual ophthalmology diagnosis data to generate actual determination data, and generating the ophthalmologic health result data including the actual ophthalmology diagnosis data and the actual determination data corresponding to the actual ophthalmology diagnosis data.

The ophthalmologic disease measuring method may include transmitting and receiving treatment management data generated in accordance with the ophthalmologic health result data with the user terminal.

The ophthalmologic disease measuring method may include transmitting and receiving the treatment management data between the user terminal and a service link terminal.

Another aspect of the present invention provides an ophthalmologic disease management method using a portable terminal and performed by a healthcare server, the ophthalmologic disease management method including generating healthcare data corresponding to ophthalmologic state measurement information of a test subject received from a user terminal, sharing the healthcare data with a service link terminal, transmitting recommendation information generated in accordance with the healthcare data on the basis of hospital information received from the service link terminal to the user terminal, transmitting and receiving appointment management information between the user terminal and the service link terminal, and generating, by the service link terminal, treatment management data in accordance with the healthcare data. The service link terminal may periodically transmit notification information about the test subject to the user terminal.

Another aspect of the present invention provides an ophthalmologic disease measuring system using a portable terminal, the ophthalmologic disease measuring system including a user terminal configured to generate ophthalmologic state measurement information including actual ophthalmology imaging information acquired from a test subject and a healthcare server configured to repeatedly learn ophthalmologic health standard data generated by matching basic ophthalmology state information to basic ophthalmology result data and analyze the ophthalmologic state measurement information to generate ophthalmologic health result data of the test subject. The healthcare server extracts an actual analysis image from the actual ophthalmology imaging information, simultaneously analyzes part-specific analysis images extracted from the actual analysis image to diagnose whether the test subject has an ophthalmologic disease and generate actual ophthalmology diagnosis data, and generates the ophthalmologic health result data including actual determination data which is generated by determining a disease-specific progression stage of the ophthalmologic disease on the basis of the actual ophthalmology diagnosis data.

The ophthalmologic disease measuring system may include a service link terminal configured to share treatment management data generated in accordance with the ophthalmologic health result data.

The ophthalmologic disease measuring system may further include a manager terminal configured to receive the ophthalmologic state measurement information from the user terminal, learn the ophthalmologic health standard data when the ophthalmologic health standard data is received from the healthcare server, and generate the ophthalmologic health result data corresponding to the ophthalmologic state measurement information.

Another aspect of the present invention provides a program stored in a computer-readable recording medium to perform the ophthalmologic disease measuring method using a portable terminal and the ophthalmologic disease management method using a portable terminal in combination with a computer which is hardware.

Other details of the present invention are included in the detailed description and the accompanying drawings.

### Advantageous Effects

According to the present invention, a current state of any of various targets of imaging, particularly, companion animals, is accurately determined through accurate ophthalmologic treatment of the companion animal. Accordingly, it is possible to maintain health of the companion animal by treating a disease of the companion animal at an early stage.

According to the present invention, during ophthalmologic treatment of a target of imaging, particularly, any of various companion animals, it is possible to determine a current state of the companion animal more accurately using a portable retina measuring device regardless of movement of the companion animal.

According to the present invention, measurement result data is generated regarding the retina of a companion animal including a high-resolution video image so that a current state of the companion animal is more accurately determined. Accordingly, it is possible to maintain health of the companion animal by treating a disease of the companion animal at an early stage.

According to the present invention, a retina measuring device is portable, and thus it is possible to improve convenience of a user as necessary regardless of time and place and also determine a current state of a companion animal more accurately by acquiring a high-resolution image. Accordingly, it is possible to improve convenience and reliability while respecting diversity of users.

According to the present invention, it is possible to acquire a high-resolution video image for accurate ophthalmologic treatment of humans as well as companion animals.

According to the present invention, when an abnormality occurs in an eye of a test subject, particularly, a companion animal, a current state of the companion animal can be quickly and accurately determined using a portable terminal. Accordingly, the caregiver of the companion animal can have confidence.

According to the present invention, a current state of the companion animal is quickly and accurately determined in real time using a portable terminal so that convenience and reliability of a user can be improved.

According to the present invention, hospital information regarding a current state of a companion animal is also provided, and thus it is possible to maintain health of the companion animal by treating a disease of the companion animal at an early stage in a hospital capable of handling the disease of the companion animal.

According to the present invention, notification information about a companion animal is continuously provided to a caregiver, it is possible to encourage the caregiver to visit a veterinary hospital again and to prevent deviation from the veterinary hospital. According to the present invention, because result data of a companion animal is shared with a linked service, it is possible to find and deal with a state of the companion animal more accurately. Accordingly, the caregiver of the companion animal can have confidence.

Effects of the present invention are not limited to those described above, and other effects which have not been described will be clearly understood by those of ordinary skill in the art from the following description.

### BRIEF DESCRIPTION OF THE DRAWINGS

FIG. 1 is a diagram illustrating a portable retina measuring device according to an embodiment of the present invention.

FIG. 2 is a detailed diagram illustrating an imaging unit shown in FIG. 1.

FIG. 3 is a diagram illustrating a light-source emitting unit shown in FIG. 2.

FIG. 4 is a conceptual diagram illustrating an ophthalmologic disease measuring system using a portable terminal according to an embodiment of the present invention.

FIG. 5 is a flowchart illustrating a method of measuring a retina of a companion animal according to an embodiment of the present invention.

FIG. 6 is a conceptual diagram illustrating an ophthalmologic disease measuring system using a portable terminal according to an embodiment of the present invention.

FIG. 7 is a diagram illustrating a detailed configuration of the ophthalmologic disease measuring system using a portable terminal shown in FIG. 6.

FIGS. 8 and 9 are diagrams illustrating a method of acquiring basic ophthalmology imaging information.

FIG. 10 is a table illustrating basic ophthalmology result data labeled with basic ophthalmology state information.

FIG. 11 is a set of graphs illustrating a method of verifying basic ophthalmology result data.

FIG. 12 is a sequence diagram illustrating an ophthalmologic disease measuring method using a portable terminal according to an embodiment of the present invention.

FIGS. 13 and 14 are detailed diagrams illustrating a method of generating ophthalmologic health standard data shown in FIG. 12.

FIGS. 15 to 18 are detailed diagrams illustrating a method of generating basic ophthalmology result data shown in FIG. 13.

FIG. 19 is a set of detailed views illustrating a method of generating ophthalmologic health result data shown in FIG. 7.

### MODE TO PRACTICE THE INVENTION

Advantages and features of the present invention and methods of achieving the same will become apparent with reference to embodiments described in detail below in conjunction with the accompanying drawings. However, the present invention is not limited to the embodiments disclosed below and may be implemented in various different forms. The embodiments are provided only to make the disclosure of the present invention complete and to fully convey the scope of the present invention to those of ordinary skill in the art to which the present invention pertains. The present invention is defined only by the scope of the claims.

Terminology used herein is for the purpose of describing the embodiments and is not intended to limit the present invention. As used herein, the singular forms include the plural forms as well unless the context clearly indicates otherwise. The terms “comprises” and/or “comprising” used herein specify the presence of the listed components and do not preclude the presence or addition of one or more components other than those listed. Throughout the specification, like reference numerals refer to like components. “And/or” includes any and all combinations of one or more of the listed components. Although “first,” “second,” etc. may be used herein to describe various components, these components are not limited by these terms. These terms are only used to distinguish one component from another. Therefore, as used herein, a first component may be a second component within the technical spirit of the present invention.

Unless otherwise defined, all terms (including technical and scientific terms) used herein have the same meaning as commonly understood by those of ordinary skill in the art to which the present invention pertains. Also, terms defined in commonly used dictionaries are not interpreted ideally or excessively unless expressly so defined herein.

Hereinafter, embodiments of the present invention will be described in detail with reference to the accompanying drawings.

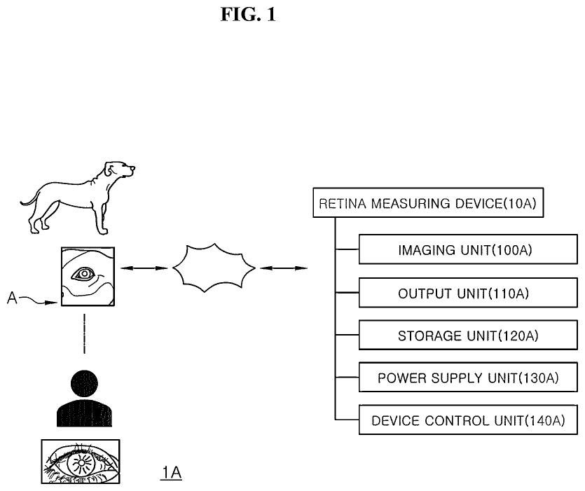

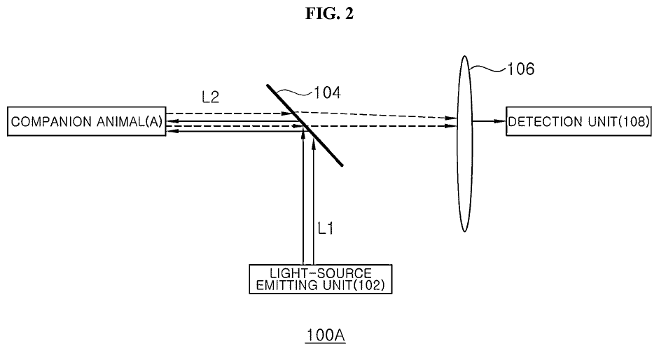

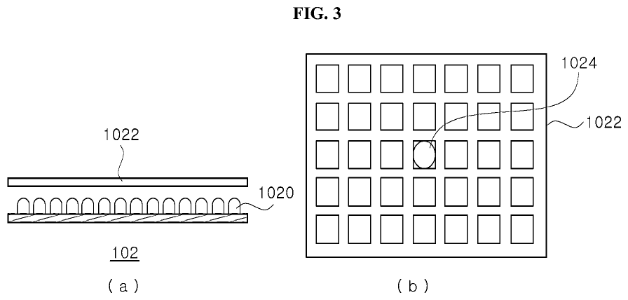

FIG. 1 is a diagram illustrating a portable retina measuring device according to an embodiment of the present invention, FIG. 2 is a detailed diagram illustrating an imaging unit shown in FIG. 1, and FIG. 3 is a diagram illustrating a light-source emitting unit shown in FIG. 2.

As shown in FIG. 1, a portable retina measuring device 10A according to the embodiment of the present invention may include an imaging unit 100A, an output unit 110A, a storage unit 120A, a power supply unit 130A, and a device control unit 140A.

First, according to the present embodiment, the portable retina measuring device 10A may be a portable device for imaging retinas of various imaging targets A but is not limited thereto. Here, an imaging target A is limited to a companion animal, particularly, a puppy, but is not limited thereto. The target of measurement may not only be retinas of various companion animals but may also be retinas of people.

According to an embodiment, the portable retina measuring device 10A may be moved in x-y-z directions and fixed to acquire photographic images in accordance with the shape or movement of the companion animal A. For example, to image a retina in accordance with a type and size of the companion animal A, the companion animal A may be fixed using a fixing member such as an elastic material band, a Velcro strap, a helmet, or a strap that fixes the head, neck, paws, body, and/or the like.

Here, the companion animal A may be fixed to the portable retina measuring device 10A to image a retina, but the present invention is not limited to this case. For example, the retina may be imaged while a tester or a caregiver holds the companion animal A.

A light-source emitting unit 102 is a radiation unit that emits light to the retina of the companion animal A. Referring to FIG. 3, the light-source emitting unit 102 may include a center of the grid 1020, a grid 1022, and an imaging element 1024. The imaging unit 100A may acquire a photographic image by imaging a state of the retina of the companion animal A which is a target of imaging. Here, the imaging unit 100A may image both retinas of the companion animal A or separately image the retina of the left eye or right eye. The photographic image may be a photograph or a video.

Specifically, the imaging unit 100A may include the light-source emitting unit 102, a reflection unit 104, a lens unit 106, and a detection unit 108.

The light-source emitting unit 102 may emit light L to the retina of the companion animal A.

Referring to FIG. 3A, the light-source emitting unit 102 may include the light source unit 1020 which generates a light source L1, the grid 1022 which is located to correspond to the light source unit 1020, and the imaging element 1024 which is located at the center of the grid 1022.

The light source unit 1020 may generate the light source L1 in various wavelength bands, and there may be a plurality of light source units 1020. The light source unit 1020 may be a light-emitting diode which emits infrared rays but is not limited thereto. The light source unit 1020 may be disposed on a substrate. According to an embodiment, a heat sink plate may be formed on a bottom surface of the substrate to quickly conduct heat generated from the light source unit 1020 to the heat sink plate. Accordingly, it is possible to minimize degradation of the light source unit 1020.

According to an embodiment, the light source unit 1020 may be located to correspond to a grid pattern of the grid 1022, but the location of the light source unit is not limited thereto. As shown in FIG. 3B, the grid 1022 may be formed in the shape of a grid plate. For example, the grid 1022 may include uniform quadrangle holes. However, the shapes of the holes are not limited thereto, and the grid 1022 may include circular or polygonal holes.

Also, the light source L1 is emitted to the retina of the companion animal A in a grid pattern in accordance with the grid shape, and thus a photographic image may be acquired from each of focuses corresponding to the grid shape.

The imaging element 1024 may be located at the center of the grid 1022 to image the retina of the companion animal A. Here, the imaging element 1024 may be a lens of a camera. The reflection unit 104 may change a path of the light source L1 emitted from the light-source emitting unit 102 by reflecting the light source L1 to the retina of the companion animal A.

Also, the reflection unit 104 may change a path of reflected light L2 reflected from the retina of the companion animal A so that the reflected light L2 is transmitted to the detection unit 108.

The lens unit 106 may be located between the reflection unit 104 and the detection unit 108 and may condense the reflected light L2 and transmit a photographic image to the detection unit 108.

According to an embodiment, the lens unit 106 may automatically adjust the focus of the reflected light L2. For example, the lens unit 106 may include a lens that performs an autofocusing actuator function.

The detection unit 108 may include a means of detection for detecting the reflected light L2 and acquiring the photographic image. For example, the detection unit 108 may be a complementary metal-oxide semiconductor (CMOS) image sensor for capturing an image when a light source in the visible light wavelength band and/or a light source in the infrared wavelength band is used.

The output unit 110A may output measurement result data corresponding to the photographic image acquired through the imaging unit 100A. For example, the output unit 110A may include a display for outputting signs, letters, numbers, etc. on a screen in accordance with the measurement result data, a lamp for outputting the same with color changes or blinking, a speaker for outputting the same in the form of audio, etc. Here, the measurement result data may include a high-resolution video image that is generated by processing and correcting a plurality of photographic images on the basis of the grid pattern.

According to an embodiment, the output unit 110A may output a current operating state of the portable retina measuring device 10A.

According to an embodiment, the output unit 110A may output an operation of the device control unit 140A to acquire a photographic image through the imaging unit 100A. For example, the output unit 110A may output a distance measurement value between the retina of the companion animal A and the imaging unit 100A and a lens focus value of the imaging element 1024 corresponding to the distance measurement value on the basis of the grid pattern.

According to an embodiment, the output unit 110A may output a plurality of photographic images acquired on the basis of the grid pattern. For example, the plurality of photographic images may include a reference image acquired centering on the retina of the companion animal A and sub-images acquired from the periphery of the retina of the companion animal A.

The storage unit 120A may store data acquired through the imaging unit 100A and data output through the output unit 110A.

The storage unit 120A may store data that supports various functions of the portable retina measuring device 10A. In other words, the storage unit 120A may store a plurality of application programs or applications run on the portable retina measuring device 10A and data and instructions for operations of the portable retina measuring device 10A. At least some of the application programs may be downloaded from an external server through wireless communication.

Under the control of the device control unit 140A, the power supply unit 130A may receive external power or internal power and supply the power to each of the components included in the portable retina measuring device 10A. The power supply unit 130A includes a battery (not shown), and the battery level may be visually checked. The battery may be connected to a 220 V commercial power source, a laptop computer, or a computer through a Universal Serial Bus (USB) cable for charging. Also, the battery unit is a cell phone battery. A 3.7 V lithium-ion battery which is the most economical and efficient secondary battery may be used to allow charging with a cell phone battery charger. Unlike this, the battery may be an embedded battery or a battery in a removable form.

The device control unit 140A controls operations of the imaging unit 100A according to a user's manual manipulation. When a photographic image is received from the imaging unit 100A, the device control unit 140A may automatically process the acquired photographic image and generate and output measurement result data.

Here, the measurement result data is a high-resolution video image acquired through the imaging unit 100A and may include information on a current retina state of the companion animal A. According to an embodiment, the measurement result data may include identification information, such as a type, a weight, a birthdate, a name, a hospital record, etc., of the companion animal.

Specifically, the device control unit 140A may calculate a distance measurement value between the companion animal A and the imaging unit 100A and control an operation of the imaging unit 100A to generate measurement result data including a high-resolution video image using an optimal image alignment algorithm and a correction algorithm for the captured photographic image. For example, the device control unit 140A may generate measurement result data of a clear high-resolution image by performing autofocus, distortion removal, illusion removal, image correction, etc. on the photographic image.

More specifically, the device control unit 140A may calculate a distance measurement value between the retina of the companion animal A and the imaging unit 100A. When the calculated distance measurement value is a preset distance measurement value, the device control unit 140A determines that the companion animal A is in a photographable state and sets an imaging time by adjusting a lens focus value. In other words, the device control unit 140A may acquire the plurality of photographic images including the reference image acquired centering on the retina of the companion animal A and sub-images acquired from the periphery of the retina of the companion animal A and generate the measurement result data of the clear high-resolution image by processing and correcting the plurality of photographic images. Here, the device control unit 140A may generate the high-resolution video image by processing and correcting the plurality of sub-images on the basis of the grid pattern included in the photographic images and the reference image.

According to an embodiment, the device control unit 140A may generate the high-resolution video image by extracting, processing, and correcting the plurality of sub-images for each of focuses in accordance with the grid shape on the basis of the reference image and the grid pattern included in the photographic images.

According to an embodiment, when the photographic images correspond to a video, the device control unit 140A may extract images from the video and generate high-resolution video images.

According to an embodiment, the device control unit 140A may generate measurement result data by supplementing the photographic images with standard result data. Here, the standard result data may include, but is not limited to, image information about the normal retina of the companion animal A and image information about various cases of ophthalmologic diseases of the companion animal A.

According to an embodiment, the device control unit 140A may generate interpretation result data regarding the measurement result data of the companion animal by comparing and analyzing the measurement result data with the standard result data. For example, the interpretation result data may include whether the companion animal A has a disease and information on the disease when the companion animal A has a disease. The types of diseases may include bacterial or inflammatory diseases, such as keratoconjunctivitis sicca, epiphora, corneal ulcer, etc., but are not limited thereto.

The portable retina measuring device 10A having the above structure is a device for imaging the retina of the companion animal A and generating measurement result data. The portable retina measuring device 10A may generate measurement result data including a high-resolution video image using a plurality of photographic images acquired from the retina of the companion animal A which is a test subject. Accordingly, a current state of the companion animal A is accurately determined through accurate ophthalmologic treatment of the companion animal A. Therefore, it is possible to maintain health of the companion animal A by treating a disease of the companion animal A at an early stage.

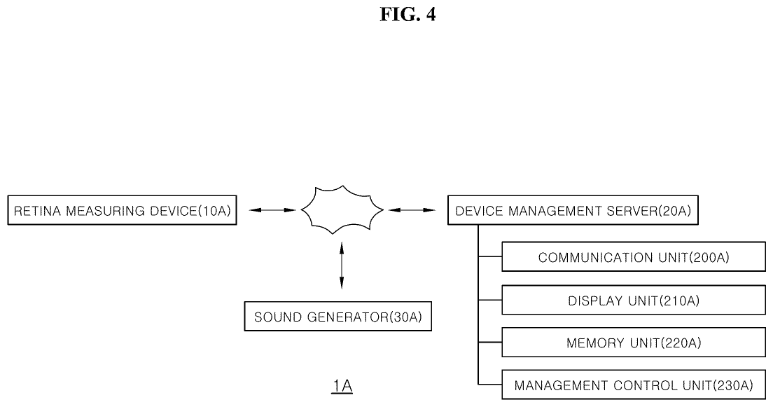

FIG. 4 is a conceptual diagram illustrating a portable retina measuring device according to another embodiment of the present invention.

As shown in FIG. 4, an ophthalmologic disease measuring system 1A using a portable terminal according to another embodiment of the present invention may include a portable retina measuring device 10A, a device management server 20A, and a sound generator 30A. Here, the sound generator 30A may be omitted.

The portable retina measuring device 10A and the device management server 20A may be synchronized in real time using a wireless communication network to transmit and receive data.

The device management server 20A may include a communication unit 200A, a display unit 210A, a memory unit 220A, and a management control unit 230A.

The communication unit 200A may transmit and receive data between the portable retina measuring device 10A and the device management server 20A.

According to an embodiment, the communication unit 200A may transmit measurement result data to a terminal or an external server.

The display unit 210A may monitor an operating state of the portable retina measuring device 10A, an operating state of the device management server 20A, data transmitted and received between the portable retina measuring device 10A and the device management server 20A, etc. caused by a user's manipulation. In other words, the operating state of the portable retina measuring device 10A is checked in real time. Accordingly, when an error or a failure occurs, a manager can quickly handle the error or failure so that the user's satisfaction of use increases more.

The memory unit 220A may store data transmitted or received through the communication unit 200A and data for supporting various functions of the portable retina measuring device 10A.

The memory unit 220A may store a plurality of application programs or applications run on the device management server 20A and data and instructions for operations of the device management server 20A. At least some of the application programs may be downloaded from an external server through wireless communication.

The management control unit 230A may generate measurement result data including a high-resolution video image using a photographic image acquired from the portable retina measuring device 10A.

According to an embodiment, the management control unit 230A may generate standard result data from the acquired photographic image or the measurement result data using a deep learning or machine learning technique. Here, the standard result data may be updated in real time in accordance with the measurement result data.

According to an embodiment, when measurement result data is received from the portable retina measuring device 10A, the management control unit 230A may generate interpretation result data regarding the received measurement result data on the basis of the standard result data.

The device management server 20A having the above structure may be a computing device implemented by a hardware circuit (e.g., a CMOS-based logic circuit), firmware, software, or a combination thereof. For example, the device management server 20A may be implemented in the form of one of various electrical structures using transistors, logic gates, and electronic circuitry.

The sound generator 30A may generate sound for stopping movement of the companion animal A and distracting the companion animal A in the case of imaging a retina of the companion animal A to acquire measurement data from the companion animal A.

Specifically, the companion animal A needs to gaze in a certain direction or forward to image the retina of the companion animal A, and thus the attention of the companion animal A may be attracted using the sound generator 30A. For example, the sound generator 30A may generate the same kind of animal sound as that of the companion animal A which is a target of imaging.

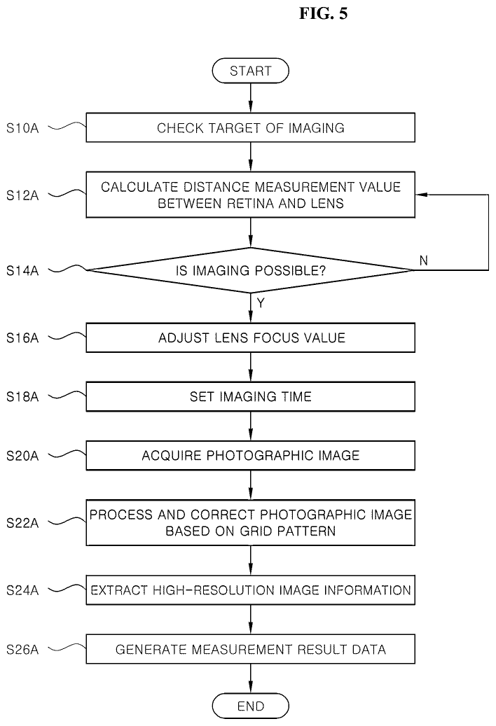

FIG. 5 is a flowchart illustrating a method of measuring a retina of a companion animal according to an embodiment of the present invention.

Referring to FIG. 5, a tester may check the companion animal A to image a retina of the companion animal A (S10A).

Here, the head of the companion animal A may be fixed and supported using a fixing device. Unlike this, however, the retina may be imaged while the tester or a caregiver holds the companion animal A.

Subsequently, the portable retina measuring device 10A may calculate a distance measurement value between the retina of the companion animal A and the imaging unit 100A (512A).

According to an embodiment, the location of the portable retina measuring device 10A may be adjusted by the tester's manual manipulation to calculate the distance measurement value of the retina of the companion animal A.

Subsequently, when the calculated distance measurement value is a preset distance measurement value, the portable retina measuring device 10A may determine that the companion animal A is in a photographable state and adjust a lens focus value (S14A and S16A).

Subsequently, the portable retina measuring device 10A may set an imaging time on the basis of the distance measurement value and the lens focus value (S18A).

Subsequently, the portable retina measuring device 10A may acquire a photographic image on the basis of the imaging time (S20A).

For example, when the distance measurement value and the lens focus value both satisfy preset conditions, the portable retina measuring device 10A may acquire five photographic image every one seconds, thereby acquiring a total of 300 photographic images in 60 seconds.

Subsequently, the portable retina measuring device 10A may extract high-resolution image information by processing and correcting the plurality of acquired photographic images on the basis of a grid pattern (S22A and S24A).

In other words, the portable retina measuring device 10A may generate a high-resolution video image by processing and correcting the plurality of photographic images including a reference image acquired centering on the retina of the companion animal A and sub-images acquired from the periphery of the retina of the companion animal A in accordance with the imaging time.

For example, the portable retina measuring device 10A may generate the high-resolution video image using an optimal image alignment algorithm and a correction algorithm for the photographic images.

Finally, the portable retina measuring device 10A may generate measurement result data of the high-resolution image (S26A).

Meanwhile, when the calculated distance measurement value is not the preset distance measurement value, the portable retina measuring device 10A may not determine that the companion animal A is in a photographable state and may calculate a new distance measurement value.

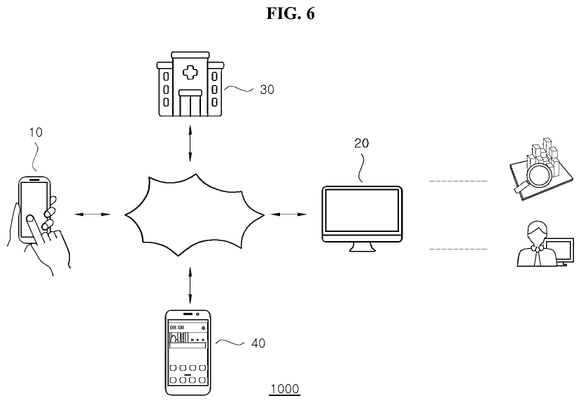

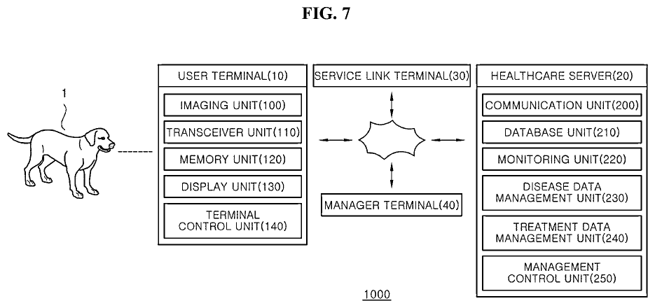

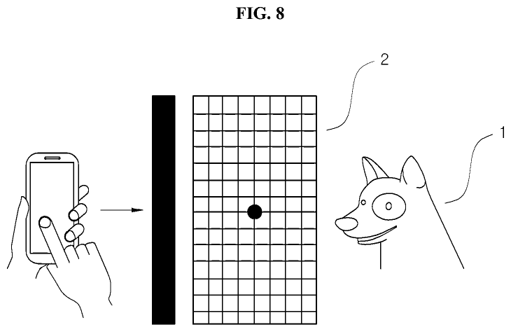

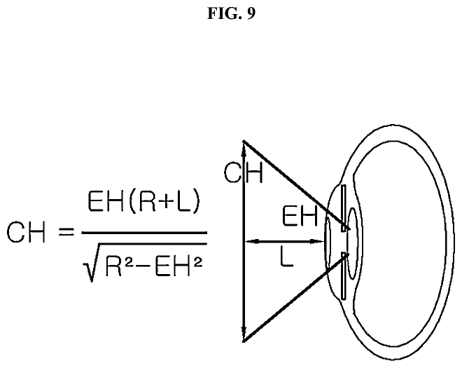

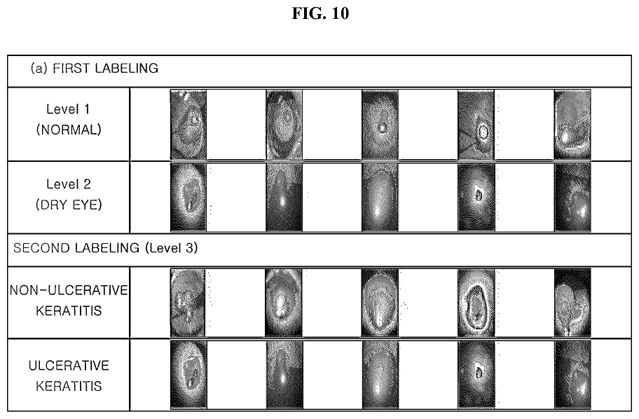

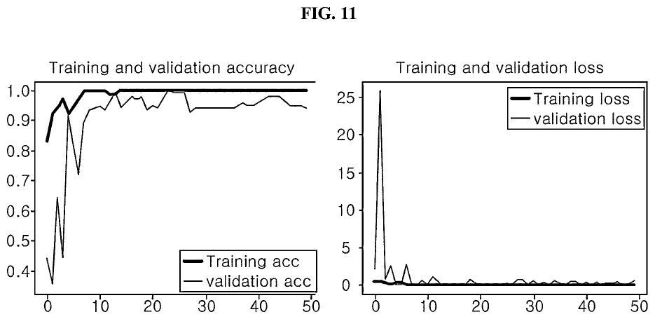

FIG. 6 is a conceptual diagram illustrating an ophthalmologic disease measuring system using a portable terminal according to an embodiment of the present invention, and FIG. 7 is a diagram illustrating a detailed configuration of the ophthalmologic disease measuring system using a portable terminal shown in FIG. 6. FIGS. 8 and 9 are diagrams illustrating a method of acquiring basic ophthalmology imaging information. FIG. 10 is a table illustrating basic ophthalmology result data labeled with basic ophthalmology state information. FIG. 11 is a set of graphs illustrating a method of verifying basic ophthalmology result data.

As shown in FIGS. 6 and 7, an ophthalmologic disease measuring system 1000 using a portable terminal according to the embodiment of the present invention may include a user terminal 10, a healthcare server 20, a service link terminal 30, and a manager terminal 40. Here, the manager terminal 40 may be omitted.

The user terminal 10, the healthcare server 20, the service link terminal 30, and the manager terminal 40 may be synchronized in real time using a wireless communication network to transmit and receive data. The wireless communication network may support various long-range communication methods. For example, various communication methods, such as Wireless Local Area Network (WLAN), Digital Living Network Alliance (DLNA), Wireless Broadband (WiBro), World Interoperability for Microwave Access (WiMAX), Global System for Mobile communication (GSM), Code Division Multiple Access (CDMA), CDMA2000, Enhanced Voice-Data Optimized or Enhanced Voice-Data Only (EV-DO), Wideband CDMA (WCDMA), High Speed Downlink Packet Access (HSDPA), High Speed Uplink Packet Access (HSUPA), the Institute of Electrical and Electronics Engineers (IEEE) 802.16, Long Term Evolution (LTE), LTE-Advanced (LTEA), Wireless Mobile Broadband Service (WMBS), Bluetooth Low Energy (BLE), ZigBee, Radio Frequency (RF), Long Range (LoRa), etc., may be applied. However, communication methods are not limited thereto, and various widely known wireless communication or mobile communication methods may be applied.

In the present embodiment, it is described that the ophthalmologic disease measuring system 1000 is used for imaging an eye of a companion animal, particularly, a puppy, and determining whether the companion animal has an ophthalmologic disease and a disease-specific progression stage when the companion animal has an ophthalmologic disease, but the present invention is not limited thereto. For example, it is possible to measure not only ophthalmologic diseases of various animals including vertebrates, such as mammals, birds, reptiles, amphibians, fish, etc., invertebrates, such as arthropods and mollusks, etc. living with a caregiver, a companion, or a dog owner (hereinafter, “caregiver”) but also ophthalmologic diseases of humans.

The user terminal 10 is a portable terminal carried by a caregiver of a companion animal 1 and may operate using an application program or application in the present disclosure. The application program may be downloaded from an external server or the hesp aalthcare server 20 through wireless communication. For example, the user terminal 10 may be one of various terminals, such as a smartphone, a personal digital assistant (PDA), a tablet, a wearable device (e.g., a smartwatch, smart glasses, a head mounted display (HMD), etc.), and various Internet of Things (IoT) terminals, but is not limited thereto.

As shown in FIG. 7, the user terminal 10 may include an imaging unit 100, a transceiver unit 110, a memory unit 120, a display unit 130, and a terminal control unit 140.

The imaging unit 100 may recognize an eyeball of the companion animal 1 using a camera (not shown) provided in the user terminal 10 to acquire actual ophthalmology imaging information captured centering on the center of the eyeball. Here, the actual ophthalmology imaging information may include information on a photograph and/or a video of the companion animal 1 in need of management. For example, the actual ophthalmology imaging information may include a photograph or a video obtained by capturing a state of the eye of the companion animal 1 but is not limited thereto.

According to an embodiment, the imaging unit 100 may image the state of the eye of the companion animal 1 using a scanner 2 which is an imaging assistant tool to be described below, to acquire the actual ophthalmology imaging information. In other words, the state of the eye of the companion animal 1 may be imaged using the scanner which projects a grid pattern onto a pupil of the companion animal 1 so that the actual ophthalmology imaging information may be acquired.

The transceiver unit 110 may transmit ophthalmologic state measurement information to the healthcare server 20 and receive healthcare data generated on the basis of ophthalmologic health standard data from the healthcare server 20. Here, the ophthalmologic health standard data may be updated in real time in accordance with ophthalmologic health result data.

Here, the ophthalmologic state measurement information may include a photograph and a video whose brightness, visibility, etc. are automatically adjusted in consideration of surroundings, shaking, blinking, whether the pupil is imaged, etc. on the basis of the actual ophthalmology imaging information.

Also, the ophthalmologic health standard data may be data generated by matching basic ophthalmology state information of the companion animal 1 to basic ophthalmology result data.

The basic ophthalmology state information may include basic information and basic ophthalmology imaging information. The basic information may include caregiver information, abandonment information, hospital record information, unique identification numbers, dog breeds, sexes, ages, weights, neutered statuses, etc. The caregiver information includes contact numbers and the like, and the hospital record information may include vaccination information, medical treatment information, allergies, etc. According to an embodiment, the hospital record information may include beauty information. The basic ophthalmology imaging information may include general imaging information captured in a general imaging mode and scanner imaging information captured using the scanner 2.

The basic ophthalmology result data may include basic diagnosis data and basic determination data. The basic diagnosis data may be data for diagnosing whether the companion animal 1 has an ophthalmologic disease using the basic ophthalmology imaging information, and the basic determination data may be data for determining a disease-specific progression stage and the like of an ophthalmologic disease on the basis of the basic diagnosis data. Here, the basic diagnosis data may be determined visually but is not limited thereto.

The ophthalmologic health result data may include actual ophthalmology diagnosis data obtained by diagnosing whether the companion animal 1 has an ophthalmologic disease using the ophthalmologic state measurement information on the basis of the ophthalmologic health standard data and actual determination data obtained by determining a disease-specific progression stage corresponding to the actual ophthalmology diagnosis data and the like on the basis of the ophthalmologic health standard data. Here, the actual ophthalmology diagnosis data may be determined visually but is not limited thereto.

The names of ophthalmologic diseases may include dry eye, ulcerative keratitis, non-ulcerative keratitis, conjunctivitis, glaucoma, uveitis, epiphora, etc., but are not limited thereto.

According to an embodiment, the transceiver unit 110 may receive the ophthalmologic health standard data from the healthcare server 20 and transmit the ophthalmologic health result data to the healthcare server 20.

According to an embodiment, when the transceiver unit 110 transmits the ophthalmologic state measurement information from the user terminal 10 to the healthcare server 20, the transceiver unit 110 may receive the ophthalmologic health result data from the healthcare server 20.

The transceiver unit 110 may transmit and receive treatment management data. Here, the treatment management data may include recommendation information which is recommendable in accordance with a current state or a disease state of the companion animal 1, appointment management information, veterinary hospital link information, and the hospital record information but is not limited thereto.

The memory unit 120 may store data transmitted or received through the transceiver unit 110 and data for supporting various functions of the user terminal 10.

The memory unit 120 may store a plurality of application programs or applications run on the user terminal 10 and data and instructions for operations of the user terminal 10. At least some of the application programs may be downloaded from the external server through wireless communication.

The display unit 130 is a means of visually and audibly outputting a current operating state of the user terminal 10 and may include a display for outputting signs, letters, numbers, etc. on a screen in accordance with the operating state, a lamp for outputting the same with color changes or blinking, a speaker for outputting the same in the form of audio, etc.

For example, in the case of outputting the ophthalmologic health result data after measurement of the companion animal 1 is finished, the display unit 130 may display the actual ophthalmology diagnosis data with O or X, blink the screen in red or green, display guide text, such as “Everything is normal,” “Please visit a veterinarian,” etc., or display actual determination data together with dictionary information. Here, the display unit 130 may output the guide text by sound so that the caregiver can accurately check the result of the companion animal 1.

Also, in the case of outputting the treatment management data, the display unit may display hospital information, such as the location, the contact number, available appointment dates, etc., of a hospital corresponding to the recommendation information generated on the basis of the veterinary hospital link information together with a road view or a map or calendar, display the hospital record information including detailed treatment information, prevention information, etc., or display appointment management information including an appointment completion signal received in response to an appointment request signal of the user terminal 10 through the screen.

The terminal control unit 140 may operate the imaging unit 100 by the caregiver's manual manipulation to generate the ophthalmologic state measurement information of the companion animal 1 and receive and output the ophthalmologic health result data for the ophthalmologic state measurement information.

Specifically, the terminal control unit 140 may recognize the eyeball of the companion animal 1 and automatically correct the actual ophthalmology imaging information acquired centering on the eyeball to generate the ophthalmologic state measurement information. Here, the ophthalmologic state measurement information may include actual basic information and the actual ophthalmology imaging information. The actual basic information may include caregiver information, the hospital record information, unique identification information, the dog breed, the sex, the age, the weight, the neutered status, etc. The caregiver information may include the contact number and the like, and the hospital record information may include the vaccination information, the medical treatment information, the allergies, etc. According to an embodiment, the hospital record information may include beauty information.

The actual ophthalmology imaging information may include a photograph and a video that are generated by automatically adjusting brightness and visibility of the actual ophthalmology imaging information in consideration of surroundings, shaking, blinking, and whether the pupil is imaged. Here, the photograph may be at least one, and the video may last at least 10 seconds, but the photograph and the video are not limited thereto.

According to an embodiment, the ophthalmologic state measurement information may include photograph and/or video information captured in the general imaging mode or photograph and/or video information captured using the scanner 2 in the scanner imaging mode.

In other words, the terminal control unit 140 may receive the ophthalmologic health result data for the ophthalmologic state measurement information regardless of time and place using the portable user terminal 10 and accurately determine whether the companion animal 1 has an ophthalmologic disease and a disease-specific progression stage of the ophthalmologic disease. Accordingly, it is possible to maintain health of the companion animal 1 by treating the ophthalmologic disease of the companion animal 1 at an early stage, and thus convenience and reliability can be improved while diversity of caregivers is respected.

According to an embodiment, when the ophthalmologic health standard data is received from the healthcare server 20, the terminal control unit 140 may generate the ophthalmologic health result data by comparing and analyzing the ophthalmologic state measurement information on the basis of the ophthalmologic health standard data.

Also, the terminal control unit 140 may transmit or receive the treatment management data corresponding to the current state or the disease state of the companion animal 1 on the basis of the ophthalmologic health result data.

Specifically, the terminal control unit 140 may generate an appointment request signal including a hospital selection, a hospital appointment request, etc. on the basis of recommendation information which is recommended in accordance with the ophthalmologic health result data on the basis of the veterinary hospital link information, transmit the appointment request signal to the healthcare server 20 or the service link terminal 30, and receive the appointment completion signal corresponding to the appointment request signal from the healthcare server 20 or the service link terminal 30. Also, the terminal control unit 140 may receive the hospital record information of the companion animal 1 from the healthcare server 20 or the service link terminal 30.

The healthcare server 20 may include a communication unit 200, a database unit 210, a monitoring unit 220, a disease data management unit 230, a treatment data management unit 240, and a management control unit 250.

The communication unit 200 may transmit the ophthalmologic health result data to the user terminal 10 when the ophthalmologic state measurement information is received from the user terminal 10.

According to an embodiment, when the communication unit 200 transmits the ophthalmologic health standard data to the user terminal 10, the communication unit may receive the ophthalmologic health result data from the user terminal 10.

Also, the communication unit 200 may transmit or receive the treatment management data between the user terminal 10 and the service link terminal 30.

According to an embodiment, the communication unit 200 may transmit and receive the treatment management data between the user terminal 10 and the service link terminal 30.

The database unit 210 may store data transmitted and received between the user terminal 10 and the service link terminal 30 through the wireless communication network. Here, the ophthalmologic health standard data may be updated in accordance with the ophthalmologic health result data and stored in real time.

The database unit 210 may store data for supporting various functions of the healthcare server 20. The database unit 210 may store a plurality of application programs or applications run on the healthcare server 20 and data and instructions for operations of the healthcare server 20. At least some of the application programs may be downloaded from the external server through wireless communication.

Meanwhile, the basic ophthalmology state information, the ophthalmologic state measurement information, the ophthalmologic health result data, and the ophthalmologic health standard data stored in the database unit 210 and used in the present embodiment may be stored in mapping tables corresponding to each other but are not limited thereto.

The monitoring unit 220 may monitor an operating state of the user terminal 10, an operating state of the healthcare server 20, data transmitted and received between the user terminal 10 and the healthcare server 20, etc. through the screen. In other words, a use state of the user terminal 10 can be checked in real time, which makes the caregiver's use convenient. Accordingly, it is possible to give more confidence to the caregiver.

The disease data management unit 230 may acquire basic ophthalmology state information from a plurality of companion animals 1 and analyze the acquired basic ophthalmology state information to generate basic ophthalmology result data. Here, the basic ophthalmology state information may be information acquired from puppies at facilities, such as abandoned dog centers, shelters, etc., but is not limited thereto.

As shown in FIG. 8, in the disease data management unit 230, basic information on the plurality of companion animals 1 may be input using a mobile terminal, parts of the companion animals 1 to be imaged may be set, and then basic ophthalmology imaging information including general imaging information captured in the general imaging mode and scanner imaging information captured in the scanner imaging mode may be acquired.

In the present embodiment, it has been described that the basic ophthalmology state information includes the basic ophthalmology imaging information of the eyes of the companion animals 1 on the basis of the basic information on the plurality of companion animals 1. However, the basic ophthalmology state information is not limited thereto and may include basic ophthalmology imaging information of various parts such as the face, the head, the abdomen, the feet, the chest, etc. Here, the basic ophthalmology imaging information may include photographs or videos.

Referring to FIG. 9, to collect basic ophthalmology imaging information of the eye part, the disease data management unit 230 may cause the pupils of the companion animals 1 to be accurately imaged in the general imaging mode and perform imaging after calculating the size of an image and an imaging distance so that the grid pattern of the scanner 2 is projected onto the pupils of the companion animals 1 in the scanner imaging mode.

Also, the disease data management unit 230 may determine whether the companion animals 1 have ophthalmologic diseases using the basic ophthalmology imaging information included in the basic ophthalmology state information and generate basic ophthalmology result data including disease-specific progression stages of the ophthalmologic diseases when it is determined that the companion animals 1 have ophthalmologic diseases. For example, referring to FIG. 10, the disease data management unit 230 may perform first labeling by analyzing whether the companion animals 1 have ophthalmologic diseases using the basic ophthalmology imaging information and classifying the eyes into level 1 (normal) or level 2 (dry eye) and perform second labeling by determining disease-specific progression stages and classifying the eyes into non-ulcerative keratitis, ulcerative keratitis, etc., thereby performing datafication.

Specifically, the disease data management unit 230 may extract and correct analyzable images by preprocessing the basic ophthalmology imaging information on the basis of the basic information of the companion animals 1, generate basic diagnosis data by extracting part-specific analysis images from the extracted analysis images and diagnosing whether the companion animals 1 have ophthalmologic diseases that are visually diagnosable, and determine disease-specific progression stages by simultaneously analyzing the part-specific analysis images on the basis of the basic diagnosis data, thereby generating basic determination data in which progression stages of a plurality of diseases are analyzed. Here, the basic diagnosis data may be data in which an abnormal symptom on the corneal surface is visually diagnosable using a grid pattern projected onto the pupil, and the basic determination data may be data generated by extracting the part-specific analysis images for analyzing a plurality of diseases from the analysis images extracted on the basis of the basic diagnosis data. For example, the disease data management unit 230 may determine that the companion animals 1 have dry eyes when the grid pattern projected onto the pupils is irregular, and determine that the companion animals 1 have corneal damage when the grid pattern is crushed, thereby generating the basic diagnosis data about whether the companion animals 1 have an ophthalmologic disease. Also, the disease data management unit 230 may simultaneously make judgments on part-specific analysis images in units of parts using a dilated convolution-based inception network (DCIN) algorithm on the basis of the basic diagnosis data, thereby more clearly generating the basic determination data including disease-specific progression stages.

Also, when the basic ophthalmology imaging information is information obtained by imaging eyes, the disease data management unit 230 may extract analyzable analysis images from the photographs or the videos included in the basic ophthalmology imaging information in consideration of surroundings, shaking, blinking, whether the pupil is imaged, etc., automatically correct brightness, visibility, etc. of the extracted analysis images, extract analysis images specific to parts, such as the pupil, the white of the eye, the eyelid, etc., for analyzing a plurality of diseases from the corrected analysis images, determine disease-specific progression stages by simultaneously analyzing the part-specific analysis images, and generate basic ophthalmology result data by performing labeling by level.

Meanwhile, when the basic ophthalmology imaging information is photographs, the disease data management unit 230 may generate basic diagnosis data and basic determination data for one photograph. Unlike this, when the basic ophthalmology imaging information is a video, the disease data management unit 230 may determine normal images in the video through a filtering operation and extract at least 10 images to generate basic diagnosis data and basic determination data. Here, a Laplace filter may be used for filtering, and normal images may be acquired by filtering an image in which the area of the eye in the total area is a certain ratio or more or shaking of the images included in the video. However, the filtering operation is not limited thereto.

The treatment data management unit 240 may manage the treatment management data transmitted and received between the user terminal 10 and the service link terminal 30 on the basis of the ophthalmologic health result data. Here, the treatment management data may include recommendation information which is recommendable in accordance with a current state or a disease state of the companion animal 1, appointment management information, veterinary hospital link information, and hospital record information.

For example, when treatment is necessary in accordance with actual ophthalmology diagnosis data, the treatment data management unit 240 may provide recommendation information generated on the basis of hospital information to the user terminal 10. Here, the recommendation information may be information for recommending hospital information corresponding to the ophthalmologic health result data using the veterinary hospital link information generated on the basis of the hospital information received from the service link terminal 30.

Also, the treatment data management unit 240 may transmit and receive appointment management information between the user terminal 10 and the service link terminal 30.

For example, the treatment data management unit 240 may transmit the appointment request signal received from the user terminal 10 to the service link terminal 30 and transmit the appointment completion signal generated in response to the appointment request signal by the service link terminal 30 to the user terminal 10.

Also, the treatment data management unit 240 may transmit the hospital record information to the user terminal 10. Here, the treatment data management unit 240 may receive the hospital record information from the service link terminal 30.

For example, after treatment of the companion animal 1 is completed, the treatment data management unit 240 may transmit hospital record information including detailed treatment information to the user terminal 10. In general, the treatment data management unit 240 may transmit hospital record information including prevention information or beauty information to the user terminal 10. Also, the treatment data management unit 240 may transmit notification information about the companion animal 1 to the user terminal 10. Here, the notification information is information generated by the service link terminal 30 and may be, but is not limited to, notification information about treatment or beauty of the companion animal 1.

According to an embodiment, the treatment data management unit 240 may share the hospital record information with another server.

The management control unit 250 may generate the ophthalmologic health standard data by matching the basic ophthalmology state information to the basic ophthalmology result data using deep learning. In the present embodiment, it has been described that deep learning is used. However, the present invention is not limited thereto, and a machine learning technique, such as random forest, support vector machine, etc., may be used. Here, the management control unit 250 may update the ophthalmologic health standard data in real time in accordance with the ophthalmologic health result data.

Specifically, the management control unit 250 may repeatedly learn the basic ophthalmology state information and the basic ophthalmology result data on the basis of a convolutional neural network (CNN) algorithm and verify suitability to generate the ophthalmologic health standard data. Here, a process of verifying the ophthalmologic health standard data may involve, but is not limited to, veterinarians and research staff of a committed research institution, for example, at least three specialists, doing cross validation on suitability as shown in FIG. 11.

Also, when the ophthalmologic state measurement information is received from the user terminal 10, the management control unit 250 may generate the ophthalmologic health result data on the basis of the ophthalmologic health standard data.

Specifically, the management control unit 250 may extract image-specific actual analysis images from actual analysis images which are extracted by preprocessing the photograph and/or the video included in the ophthalmologic state measurement information and generate the ophthalmologic health result data including the actual ophthalmology diagnosis data from which whether the companion animal 1 has an ophthalmologic disease is visually diagnosable and the actual determination data which is generated by simultaneously analyzing a plurality of diseases from the part-specific actual analysis images on the basis of the actual ophthalmologic diagnosis data and determining a disease-specific progression stage corresponding to the actual ophthalmology diagnosis data.

For example, when the actual ophthalmology imaging information is information obtained by imaging the eye, the management control unit 250 may extract an analyzable actual analysis image from the photograph or the video included in the actual ophthalmology imaging information in consideration of surroundings, shaking, blinking, whether the pupil is imaged, etc., automatically correct brightness, visibility, etc. of the extracted actual analysis image, extract analysis images specific to parts, such as the pupil, the white of the eye, the eyelid, etc., for analyzing a plurality of diseases from the corrected actual analysis image, generate the actual ophthalmology diagnosis data by diagnosing whether the companion animal 1 has an ophthalmologic disease that is visually diagnosable, and determine the actual determination information by analyzing the part-specific analysis images on the basis of the actual ophthalmology diagnosis data and determining a disease-specific progression stage. In other words, the management control unit 250 may determine whether there is a scar in an actual analysis image of the pupil part, determine the color of the white of the eye, the thickness of capillaries, a red area in the white of the eye, etc. in an actual analysis image of the white of the eye, and simultaneously analyze whether the eyelash is abnormal and how rough the eyelid is in an actual analysis image of the eyelid part, thereby analyzing progression stages of a plurality of diseases through the part-specific analysis images.

Meanwhile, when the actual ophthalmology imaging information is photographs, the management control unit 250 may generate actual ophthalmology diagnosis data and actual determination data regarding one photograph. Unlike this, when the actual ophthalmology imaging information is a video, the management control unit 250 may determine normal images in the video through a filtering operation and extract at least 10 images to generate actual ophthalmology diagnosis data and actual determination data. Here, a Laplace filter may be used for filtering, and normal images may be acquired by filtering an image in which the area of the eye in the total area is a certain ratio or more or shaking of the images included in the video. However, the filtering operation is not limited thereto.

According to an embodiment, when the management control unit 250 transmits the ophthalmologic health standard data to the user terminal 10, the management control unit 250 may receive ophthalmologic health result data corresponding to the ophthalmologic state measurement data of the companion animal 1.

According to an embodiment, the management control unit 250 transmits or receives advertising information together with data transmitted to or received from the user terminal 10, the service link terminal 30, and/or the manager terminal 40. Accordingly, advertising revenue can be generated to support facilities such as abandoned dog centers, shelters, etc.

The healthcare server 20 having the above structure may automatically extract a diagnosis part from ophthalmologic state measurement information acquired through the user terminal 10 on the basis of ophthalmologic health standard data which is verified by repeatedly learning basic ophthalmology result data labeled in accordance with basic ophthalmology state information acquired from the plurality of companion animals 1, compare and analyze extracted images to analyze a plurality of diseases, and generate ophthalmologic health result data including whether the companion animal 1 has an ophthalmologic disease and a disease-specific progression stage. Accordingly, it is possible to solve problems including unnecessary hospital visits, negligence, etc. which may be caused when the state of the companion animal 1 is only visually determined.

Also, the healthcare server 20 provides recommendation information to the user terminal 10 in accordance with the current state or the disease state of the companion animal 1 so that the companion animal 1 can be quickly and accurately managed.

The healthcare server 20 may be implemented as a hardware circuit (e.g., a CMOS-based logic circuit), firmware, software, or a combination thereof. For example, the healthcare server 20 may be implemented in the form of one of various electrical structures using transistors, logic gates, and electronic circuitry.

The service link terminal 30 is a plurality of veterinary hospitals for managing health of the companion animal 1 and examining the companion animal 1 and may examine the companion animal 1 more quickly using the ophthalmologic health result data.

The service link terminal 30 may share the hospital record information with the user terminal 10, the healthcare server 20, and an additional server.

The service link terminal 30 may provide the hospital information and notification information to the user terminal 10 and/or the healthcare server 20.

According to an embodiment, the service link terminal 30 may include additional facilities such as an abandoned dog center, a shelter, etc.

The manager terminal 40 is a terminal carried by a manager and may be synchronized in real time with the user terminal 10, the healthcare server 20, and the service link terminal 30 using the wireless communication network to transmit and receive data. Here, the manager terminal 40 may transmit and receive data using an application program or an application.

The manager terminal 40 may learn the ophthalmologic health standard data received from the healthcare server 20 and analyze the ophthalmologic state measurement information received from the user terminal 10, thereby generating the ophthalmologic health result data including the actual ophthalmology diagnosis data and the actual determination data.

According to an embodiment, when the ophthalmologic state measurement information is received from the user terminal, the manager terminal 40 may compare and analyze the ophthalmologic state measurement information on the basis of the ophthalmologic health standard data to generate the ophthalmologic health result data.

According to an embodiment, when the ophthalmologic health result data is generated by the user terminal 10, the manager terminal 40 may receive the ophthalmologic health result data from the user terminal 10 and transmit the ophthalmologic health result data to the healthcare server 20. Also, when the ophthalmologic health result data is generated by the healthcare server 20, the manager terminal 40 may receive the ophthalmologic health result data from the healthcare server 20 and transmit the ophthalmologic health result data to the user terminal 10.

According to an embodiment, the manager terminal 40 may transmit and receive the treatment management data corresponding to the current state or the disease state of the companion animal 1 based on the ophthalmologic health result data with at least one of the user terminal 10, the healthcare server 20, and the service link terminal 30.

The manager terminal 40 may be one of various portable electronic communication devices that support communication with the user terminal 10, the healthcare server 20, and the service link terminal 30. For example, the manager terminal 40 may be one of various terminals, such as a smartphone, a PDA, a tablet, a wearable device (e.g., a smartwatch, smart glasses, an HMD, etc.), and various IoT terminals, but is not limited thereto.

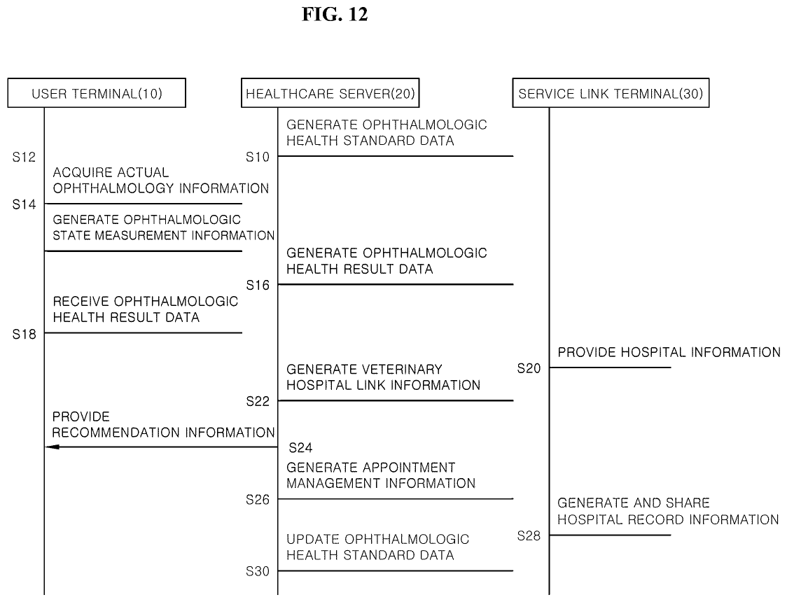

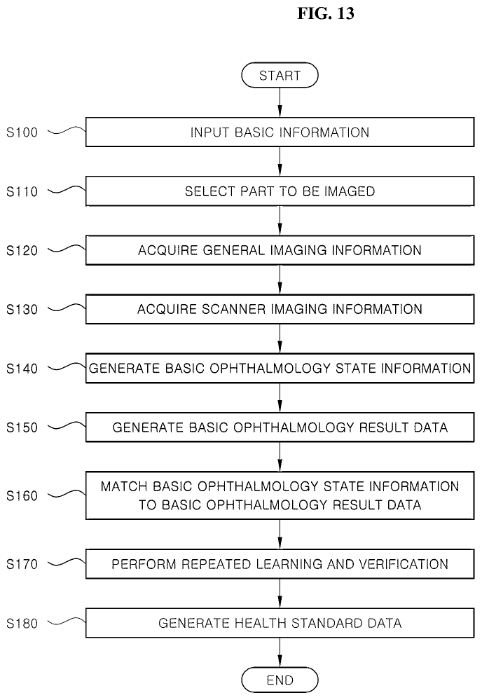

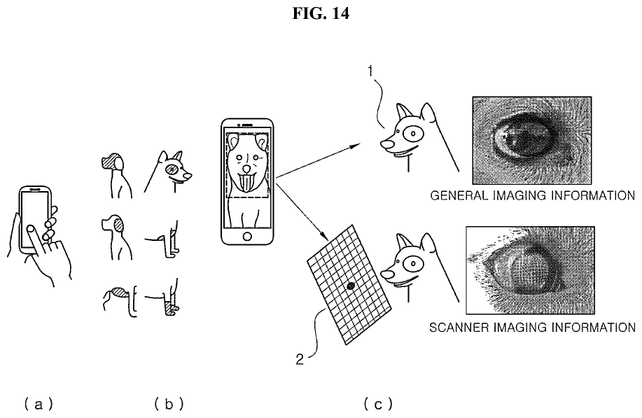

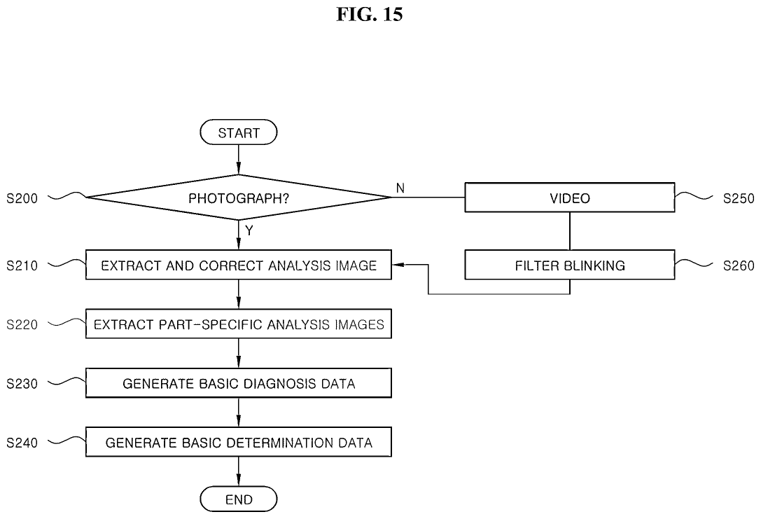

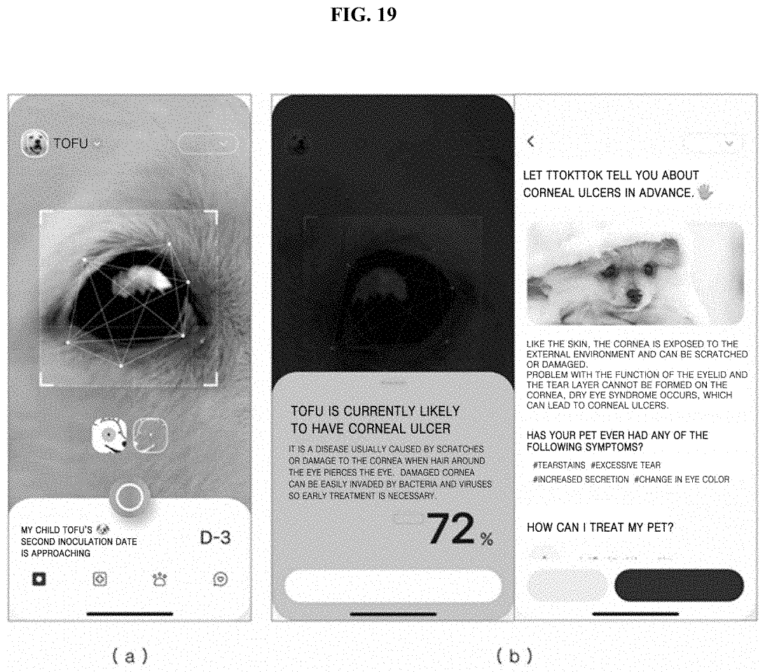

The ophthalmologic disease measuring system using a portable terminal and having the above structure according to the embodiment of the present invention operates as follows. FIG. 12 is a sequence diagram illustrating an ophthalmologic disease measuring method using a portable terminal according to an embodiment of the present invention, and FIGS. 13 and 14 are detailed diagrams illustrating a method of generating ophthalmologic health standard data shown in FIG. 12. FIGS. 15 to 18 are detailed diagrams illustrating a method of generating basic ophthalmology result data shown in FIG. 13, and FIG. 19 is a set of detailed views illustrating a method of generating ophthalmologic health result data shown in FIG. 7.

First, in the embodiment of the present invention, the companion animal 1 is described as a puppy but is not limited thereto.

As shown in FIG. 12, the healthcare server 20 may generate ophthalmologic health standard data (S10).

Specifically, referring to FIG. 13, the healthcare server 20 may acquire basic information from a plurality of companion animals 1 (S100). Here, the basic information may include caregiver information, abandonment information, hospital record information, unique identification numbers, dog breeds, sexes, ages, weights, neutered statuses, etc. but is not limited thereto.

For example, referring to FIG. 14A, the disease data management unit 230 may receive the basic information of the companion animals 1 using a separate mobile terminal.

Subsequently, the healthcare server 20 may select parts of the companion animals to be imaged on the basis of the basic information (S110).

For example, referring to FIG. 14B, the disease data management unit 230 may set the parts of the companion animals to be imaged. In other words, the disease data management unit 230 may select various body parts, such as faces, ears, abdomens, chests, etc., of the companion animals 1.

Subsequently, in the case of imaging eyes, the healthcare server 20 may acquire general imaging information first by imaging the eyes of the companion animals 1 in the general imaging mode (S120).

For example, referring to FIG. 14C, the disease data management unit 230 may image the eyes of the companion animals 1 in the general imaging mode using the mobile terminal.

Subsequently, in the case of imaging eyes, the healthcare server may acquire scanner imaging information second by imaging the eyes of the companion animals 1 in the scanner imaging mode (S130).

For example, referring to FIG. 14C, the disease data management unit 230 may image the eyes of the companion animals 1 in the scanner imaging mode using the mobile terminal and the scanner 2 having a grid pattern. Here, the scanner 2 may be disposed at the eyes of the companion animals 1 so that the grid pattern is projected onto the pupils of the companion animals 1.

Subsequently, the healthcare server 20 may generate basic ophthalmology state information using basic ophthalmology imaging information including the basic information, the general imaging information, and the scanner imaging information (S140). Here, the general imaging information and the scanner imaging information may include at least one photograph and a video lasting at least 10 seconds.

Subsequently, the healthcare server 20 may extract analyzable analysis images from the basic ophthalmology imaging information and generate basic ophthalmology result data (S150).

Specifically, as shown in FIG. 15, when information included in the basic ophthalmology imaging information is photographs (S200), the healthcare server 20 may verify whether the photographs are analyzable images and extract analysis images (S210).

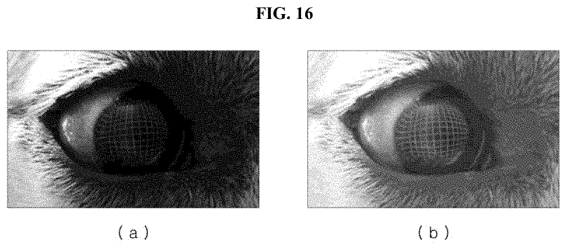

For example, the disease data management unit 230 may extract the analyzable analysis image in consideration of surroundings, shaking, blinking, whether the pupil is imaged, etc. Here, the extracted analysis images (see FIG. 16A) may be corrected (see FIG. 16B). In other words, the disease data management unit 230 may extract the periphery of the eye from the analysis images and correct white balance and brightness.

Subsequently, the healthcare server 20 may extract part-specific analysis images for analyzing a plurality of diseases from the extracted analysis images (S220).

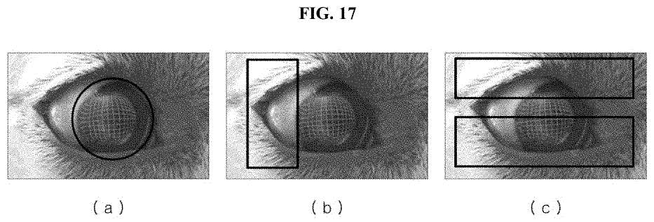

For example, referring to FIG. 17, the disease data management unit 230 may extract part-specific analysis images of the pupil part (see FIG. 17A), part-specific analysis images of the white of the eye (see FIG. 17B), and part-specific analysis images of the eyelid part from the analysis images and simultaneously analyze a plurality of diseases.

Subsequently, basic diagnosis data in which whether the companion animals have an ophthalmologic disease is diagnosed may be generated using the part-specific analysis images (S230).

For example, the disease data management unit 230 may determine that the companion animals 1 have dry eyes when the grid pattern projected onto the pupils is irregular, and determine that the companion animals 1 have corneal damage when the grid pattern is crushed, thereby generating the basic diagnosis data about whether the companion animals 1 have ophthalmologic diseases.

Subsequently, the healthcare server 20 may make judgments on the part-specific analysis images for analyzing a plurality of diseases on the basis of the basic diagnosis data and generate basic determination data (S240).

Specifically, the disease data management unit 230 may determine whether there is a scar in images of the pupil parts, determine the color of the whites of the eyes, the thickness of capillaries, a red area in the whites of the eyes, etc. in images of the whites of the eyes, and simultaneously analyze whether the eyelashes are abnormal and how rough the eyelids are in images of the eyelid parts, thereby analyzing a plurality of diseases through the part-specific analysis images.

Here, the disease data management unit 230 may simultaneously make judgments on the part-specific analysis images in units of parts using a DCIN algorithm on the basis of the basic diagnosis data, thereby more clearly generating the basic determination data in which a plurality of diseases are analyzed. However, a method for the disease data management unit 230 to generate basic determination data is not limited thereto.

Meanwhile, when information included in the basic ophthalmology imaging information is a video (S250), the disease data management unit 230 may filter out blinking in the video to extract normal images (S260).

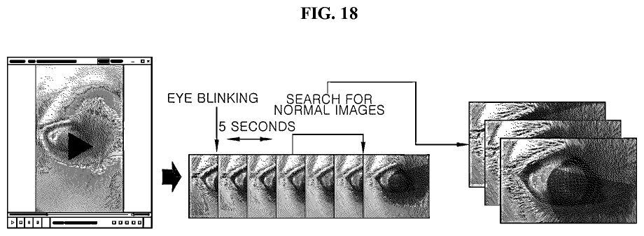

For example, referring to FIG. 18, the disease data management unit 230 may determine normal images in the video through the filtering operation to extract at least images. Here, a Laplace filter may be used for filtering, and normal images may be acquired by filtering an image in which the area of the eye in the total area is a certain ratio or more or shaking of images included in the video.

In this way, the healthcare server 20 may generate basic ophthalmology result data corresponding to the basic ophthalmology state information.

Subsequently, the healthcare server 20 may match the basic ophthalmology state information to the basic ophthalmology result data (S160) and repeatedly learn the basic ophthalmology state information and the basic ophthalmology result data on the basis of a CNN algorithm and verify suitability to generate ophthalmologic health standard data (S170 and S180).

Subsequently, when a caregiver requests a diagnosis of a current state or a disease state of a companion animal 1, the user terminal 10 may acquire actual ophthalmology imaging information of the eye of the companion animal 1 as shown in FIG. 19A (S12).

Subsequently, the user terminal 10 may receive actual basic information and generate ophthalmologic state measurement information using the actual basic information and the actual ophthalmology imaging information (S14).

Subsequently, the healthcare server 20 may generate ophthalmologic health result data corresponding to the ophthalmologic state measurement information on the basis of the ophthalmologic health standard data (S16).

Specifically, the management control unit 250 may extract and correct actual analysis images by preprocessing a photograph and/or a video included in the ophthalmologic state measurement information, extract part-specific actual analysis images from the corrected actual analysis images, and generate ophthalmologic health result data including actual ophthalmology diagnosis data from which whether there is an ophthalmologic disease in the part-specific actual analysis images is visually diagnosable and actual determination data which is generated by determining a disease-specific progression stage corresponding to the actual ophthalmology diagnosis data.

Subsequently, the user terminal 10 may receive the ophthalmologic health result data corresponding to the ophthalmologic state measurement information from the healthcare server 20 (S18).

For example, in the case of outputting the ophthalmologic health result data after ophthalmologic disease measurement of the companion animal 1 is finished, the display unit 130 may display the ophthalmology health result data as shown in FIG. 19B.

Subsequently, the service link terminal 30 may provide hospital information on the basis of the ophthalmologic health standard data (S20).

Here, the operation of providing the hospital information may be performed earlier but is not limited thereto.

Subsequently, the healthcare server 20 may generate veterinary hospital link information on the basis of the hospital information (S22).

Here, the operation of generating veterinary hospital link information may be performed earlier but is not limited thereto.

Subsequently, the healthcare server 20 may generate recommendation information corresponding to the ophthalmologic health result data on the basis of the veterinary hospital link information and transmit the recommendation information to the user terminal 10 (S24).

Subsequently, the healthcare server 20 may generate appointment management information (S26).

For example, the healthcare server 20 may receive an appointment request signal generated in accordance with customized information from the user terminal 10, receive appointment management information corresponding to the appointment request signal from the service link terminal 30, and transmit the appointment management information to the user terminal 10.

Subsequently, the service link terminal 30 may generate and share hospital record information including detailed treatment information regarding the companion animal 1 (S28).

Here, the hospital record information may be transmitted to the user terminal and the healthcare server 20.

Finally, the healthcare server 20 may update the ophthalmologic health standard data in real time in accordance with the ophthalmologic health result data (S30).

Operations of a method or algorithm described in connection with embodiments of the present invention may be directly implemented by hardware, implemented as a software unit which is executed by hardware, or implemented by a combination thereof. The software unit may be on a random access memory (RAM), a read only memory (ROM), an erasable programmable ROM (EPROM), an electrically erasable programmable ROM (EEPROM), a flash memory, a hard disk, a detachable disk, a compact disc (CD)-ROM, or any form of computer-readable recording medium well known in the technical field to which the present invention pertains.

Although embodiments of the present invention have been described with reference to the accompanying drawings, those of ordinary skill in the art to which the present invention pertains should understand that the present invention can be implemented in other specific forms without changing the technical spirit or essential characteristics thereof. Therefore, it should be understood that the above-described embodiments are illustrative in all aspects and not restrictive.

1. A portable retina measuring device comprising:
an imaging unit configured to acquire a photographic image by imaging a retina of a target of imaging; and
a device control unit configured to generate measurement result data using the acquired photographic image,
wherein the imaging unit comprises a plate-shaped light-source emitting unit including:
a light source unit configured to generate a light source for the target of imaging;
a grid located to face the light source unit and having a grid pattern; and
an imaging element located at a center of the grid.

2. The portable retina measuring device of claim 1, wherein the device control unit adjusts an imaging time using a distance measurement value between the target of imaging and the imaging unit.

3. The portable retina measuring device of claim 2, wherein the device control unit adjusts a lens focus value of the imaging element in accordance with a position of the retina of the target of imaging on the basis of the grid pattern.

4. The portable retina measuring device of claim 3, wherein, when the distance measurement value and the lens focus value are both satisfactory, the device control unit acquires a plurality of photographic images and generates the measurement result data including a high-resolution video image by processing and correcting the acquired photographic images on the basis of the grid pattern.

5. The portable retina measuring device of claim 4, wherein the photographic images include a reference image acquired centering on the retina of the target of imaging and sub-images acquired from the periphery of the retina of the target of imaging.

6. The portable retina measuring device of claim 1, further comprising a device management server configured to generate standard result data on the basis of the measurement result data and generate interpretation result data regarding the retina of the target of imaging by comparing and analyzing the standard result data and the measurement result data.

7. The portable retina measuring device of claim 1, further comprising a sound generator configured to generate, when the target of imaging is a companion animal, a sound related to the companion animal.

8. An ophthalmologic disease measuring method using a portable terminal, the ophthalmologic disease measuring method comprising:
matching, by a healthcare server, basic ophthalmology state information to basic ophthalmology result data to generate ophthalmologic health standard data;
receiving, by the healthcare server, ophthalmologic state measurement information of a test subject from a user terminal; and
comparing and analyzing, by the healthcare server, the ophthalmologic state measurement information on the basis of the ophthalmologic health standard data to generate ophthalmologic health result data,
wherein the generation of the ophthalmologic health standard data comprises:
preprocessing, by the healthcare server, images included in basic ophthalmology imaging information included in the basic ophthalmology state information;
extracting, by the healthcare server, analysis images from the preprocessed images;
extracting, by the healthcare server, part-specific analysis images from the analysis images and diagnosing whether there is an ophthalmologic disease in the part-specific analysis images to generate basic diagnosis data;
determining, by the healthcare server, disease-specific progression stages for the part-specific analysis images on the basis of the basic diagnosis data to generate basic determination data; and
generating, by the healthcare server, the basic ophthalmology result data including the basic diagnosis data and the basic determination data corresponding to the basic diagnosis data.

9. The ophthalmologic disease measuring method of claim 8, wherein the healthcare server simultaneously analyzes the part-specific analysis images on the basis of basic information of the test subject included in the basic ophthalmology state information to classify the basic ophthalmology imaging information according to the disease-specific progression stages.

10. The ophthalmologic disease measuring method of claim 8, wherein the acquiring of the basic ophthalmology imaging information comprises:
imaging, by the healthcare server, an eye of the test subject in a general imaging mode to acquire general imaging information; and
imaging, by the healthcare server, the eye of the test subject in a scanner imaging mode in which a scanner is used so that a pupil of the test subject is scanned in a grid pattern, to acquire scanner imaging information,
wherein the basic ophthalmology imaging information includes a photograph or a video.

11. The ophthalmologic disease measuring method of claim 10, further comprising a filtering operation of determining, when the basic ophthalmology imaging information is the video, normal images from the video and extracting at least ten images.

12. The ophthalmologic disease measuring method of claim 10, wherein, when it is determined that there is an abnormal symptom in a corneal surface using the grid pattern in which the pupil of the test subject is scanned, diagnosing, by the healthcare server, the test subject with the ophthalmologic disease and determining a disease-specific progression stage for the test subject.

13. The ophthalmologic disease measuring method of claim 8, wherein the generation of the ophthalmologic health standard data comprises repeatedly learning, by the healthcare server, the basic ophthalmology result data corresponding to the basic ophthalmology state information to verify the basic ophthalmology result data.

14. The ophthalmologic disease measuring method of claim 8, wherein the generation of the ophthalmologic health result data comprises:
extracting, by the healthcare server, an actual analysis image from actual ophthalmology imaging information included in the ophthalmologic state measurement information of the test subject;
extracting, by the healthcare server, part-specific actual analysis images from the actual analysis image to diagnose whether there is an ophthalmologic disease in the part-specific actual analysis images and generate actual ophthalmology diagnosis data;
determining, by the healthcare server, a disease-specific progression stage for the part-specific actual analysis images on the basis of the actual ophthalmology diagnosis data to generate actual determination data; and
generating, by the healthcare server, the ophthalmologic health result data including the actual ophthalmology diagnosis data and the actual determination data corresponding to the actual ophthalmology diagnosis data.

15. The ophthalmologic disease measuring method of claim 8, comprising transmitting and receiving, by the healthcare server, treatment management data generated in accordance with the ophthalmologic health result data with the user terminal.

16. The ophthalmologic disease measuring method of claim 15, comprising transmitting and receiving, by the healthcare server, the treatment management data between the user terminal and a service link terminal.

17. An ophthalmologic disease management method using a portable terminal, the ophthalmologic disease management method comprising:
generating, by a healthcare server, healthcare data corresponding to ophthalmologic state measurement information of a test subject received from a user terminal;
sharing, by the healthcare server, the healthcare data with a service link terminal;
transmitting, by the healthcare server, recommendation information generated in accordance with the healthcare data on the basis of hospital information received from the service link terminal to the user terminal;
transmitting and receiving, by the healthcare server, appointment management information between the user terminal and the service link terminal; and
generating, by the service link terminal, treatment management data in accordance with the healthcare data,
wherein the service link terminal periodically transmits notification information about the test subject to the user terminal.

18.-21. (canceled)
